(19) United States
(12) Patent Application Publication
(10) Pub. No.: US 2017/0112356 A1
(43) Pub. Date: Apr. 27, 2017

(54) IMAGE PROCESSING APPARATUS, IMAGE PROCESSING METHOD, COMPUTER-READABLE RECORDING MEDIUM, AND ENDOSCOPE SYSTEM

(71) Applicant: OLYMPUS CORPORATION, Tokyo (JP)

(72) Inventors: Masanori MITSUI, Tokyo (JP); Takuji HORIE, Sagamihara-shi (JP)

(73) Assignee: OLYMPUS CORPORATION, Tokyo (JP)

(21) Appl. No.: 15/398,880

(22) Filed: Jan. 5, 2017

Related U.S. Application Data
(63) Continuation of application No. PCT/JP2015/067928, filed on Jun. 22, 2015.

(30) Foreign Application Priority Data
Jul. 11, 2014 (JP) 2014-143676

(51) Int. Cl. A61B 1/00; A61B 1/06; G06T 7/90; G02B 23/24; G06T 7/00; A61B 1/04; H04N 7/18

(52) U.S. Cl. CPC A61B 1/00009; A61B 1/0005; A61B 1/04; A61B 1/0653; H04N 7/183; G02B 23/2446; G06T 7/0016; G06T 7/90; G06T 2207/10024; G06T 2207/10068; G06T 2207/30096; G06T 2207/10004; G06T 2207/30196

(57) ABSTRACT

An image processing apparatus is provided in an imaging system having an image capturing unit. The image capturing unit is configured to irradiate a subject with light and to generate first image data representing a first image based on the light reflected from the subject and having first spectral characteristics, and to generate second image data representing a second image based on the light reflected from the subject and having second spectral characteristics different from the first spectral characteristics. The image processing apparatus includes: a computing unit that determines a degree of correlation between the first image and the second image; and a control unit that causes the image capturing unit to generate the first image data at a preset frame rate, and controls timing to generate the second image data instead of the first image data based on a determination result of the degree of correlation.

### CROSS REFERENCES TO RELATED APPLICATIONS

This application is a continuation of PCT international application Ser. No. PCT/JP2015/067928, filed on Jun. 22, 2015 which designates the United States, incorporated herein by reference, and which claims the benefit of priority from Japanese Patent Application No. 2014-143676, filed on Jul. 11, 2014, incorporated herein by reference.

### BACKGROUND

1. Technical Field

The disclosure relates to an image processing apparatus, an image processing method, a computer-readable recording medium, and an endoscope system which are configured to perform image processing on a plurality of kinds of images generated by performing imaging using light having different spectral characteristics.

2. Related Art

In recent years, in the fields of an endoscope, a microscope, and the like, utilized for diagnosis are not only a normal light image that is an image generated by imaging with use of white light also called as normal light but also a special light image that is an image generated by so-called special light imaging that is imaging with use of light having specific spectral characteristics and also called as special light.

However, since a wavelength band of the special light is limited relative to the normal light, color balance of a special light image is significantly different compared with the normal light image. Therefore, the comparative observation may be hardly performed for both images.

To address such a situation, a technique is disclosed in JP 2010-172673 A in which, for example, one set of a normal light image and a special light image is generated by executing normal light imaging and special light imaging, a lesion candidate that is a portion suspected as a lesion is detected by analyzing the special light image out of these images, and the normal light image and the lesion candidate detected from the special light image corresponding to the normal light image are simultaneously displayed on a monitor.

Furthermore, a technique is disclosed in JP 2012-170640 A, in which first irradiating operation to perform irradiation with narrow band light at maximum intensity and second irradiating operation to perform irradiation with narrow band light at normal intensity are alternately repeated in every accumulation period of a CCD, a capillary component of a mucous membrane surface layer is extracted from a G pixel value obtained in the first irradiating operation by executing correlative calculation with an R pixel value obtained in the first irradiating operation, a B pixel value obtained from the second irradiating operation and the extracted component are allocated to B and G channels of a monitor, and also the G pixel value obtained from the second irradiating operation is allocated to an R channel, thereby making a monitor display a highlighted image in which the capillary is colored in reddish brown.

Furthermore, a technique is disclosed in JP 2011-160848 A, in which a region of interest in a special light image is detected based on a characteristic amount of a pixel inside the special light image, setting processing for a lapse time is performed based on a detection result of the region of interest, and display form setting processing is performed based on this lapse time for a display image formed based on a normal light image. In this Patent Literature 3, the normal light image and the special light image are obtained in a predetermined cycle, and also a highly-qualified normal light image is obtained by suppressing degradation of temporal resolution of the normal light image to be a base by increasing an obtaining rate of the normal light image more than that of the special light image.

### SUMMARY

In some embodiments, an image processing apparatus is provided in an imaging system having an image capturing unit. The image capturing unit is configured to irradiate a subject with light and to generate first image data representing a first image based on the light reflected from the subject and having first spectral characteristics, and to generate second image data representing a second image based on the light reflected from the subject and having second spectral characteristics different from the first spectral characteristics, the image processing apparatus being configured to perform image processing on the first image and the second image. The image processing apparatus includes: a computing unit configured to determine a degree of correlation between the first image and the second image; and a control unit configured to cause the image capturing unit to generate the first image data at a preset frame rate, and configured to control timing to generate the second image data instead of the first image data based on a determination result of the degree of correlation.

In some embodiments, an image processing method includes: irradiating a subject with light and generating image data representing a first image based on the light reflected from the subject and having first spectral characteristics; irradiating the subject with light and generating image data representing a second image based on the light reflected from the subject and having second spectral characteristics different from the first spectral characteristics; determining a degree of correlation between the first image and the second image; and causing the first image data to be generated at a preset frame rate, and controlling timing to generate the second image data instead of the first image data based on a determination result of the degree of correlation.

In some embodiments, provided is a non-transitory computer-readable recording medium with an executable image processing program stored thereon. The program causes a computer to execute: irradiating a subject with light and generating image data representing a first image based on the light reflected from the subject and having first spectral characteristics; irradiating the subject with light and generating image data representing a second image based on the light reflected from the subject and having second spectral characteristics different from the first spectral characteristics; determining a degree of correlation between the first image and the second image; and causing the first image data to be generated at a preset frame rate, and controlling timing to generate the second image data instead of the first image data based on a determination result of the degree of correlation.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram illustrating an imaging system including an image processing apparatus according to a first embodiment of the present invention;

FIG. 2 is a flowchart illustrating operation of the imaging system illustrated in FIG. 1;

FIG. 3 is a schematic diagram illustrating an image sequence sequentially generated by the imaging system illustrated in FIG. 1;

FIG. 4 is a schematic diagram illustrating exemplary display of a normal light image and a special light image in a lesion region extraction mode;

FIG. 5 is a schematic diagram illustrating different exemplary display of a normal light image and a special light image in the lesion region extraction mode;

FIG. 6 is a schematic diagram illustrating an image sequence sequentially formed in a modified example 1-3 of the first embodiment of the present invention;

FIG. 7 is a block diagram illustrating a configuration of an imaging system including an image processing apparatus according to a modified example 1-4 of the first embodiment of the present invention;

FIG. 8 is a block diagram illustrating a configuration of an imaging system including an image processing apparatus according to a second embodiment of the present invention;

FIG. 9 is a flowchart illustrating operation of the imaging system illustrated in FIG. 8;

FIG. 10 is a schematic diagram illustrating an image sequence sequentially generated by the imaging system illustrated in FIG. 8;

FIG. 11 is a schematic diagram illustrating exemplary display of a region of interest extracted from a normal light image and a special light image in the lesion region extraction mode;

FIG. 12 is a schematic diagram illustrating different exemplary display of a region of interest extracted from a normal light image and a special light image in the lesion region extraction mode;

FIG. 13 is a schematic diagram illustrating an image sequence sequentially formed in a third embodiment of the present invention;

FIG. 14 is a schematic diagram illustrating an image sequence sequentially formed in a fourth embodiment of the present invention;

FIGS. 15A and 15B are graphs illustrating exemplary spectral characteristics of light used in the fourth embodiment of the present invention;

FIG. 16 is a schematic diagram illustrating a different example of the image sequence sequentially formed in the fourth embodiment of the present invention;

FIG. 17 is a schematic diagram illustrating another different example of the image sequence sequentially formed in the fourth embodiment of the present invention; and

FIG. 18 is a schematic diagram illustrating an outline structure of an endoscope system according to a fifth embodiment of the present invention.

### DETAILED DESCRIPTION

In the following, an image processing apparatus, an image processing method, an image processing program, and an endoscope system according to embodiments of the present invention will be described with reference to the drawings. The present invention is not intended to be limited by these embodiments. The same reference signs are used to designate the same elements throughout the drawings.

### First Embodiment

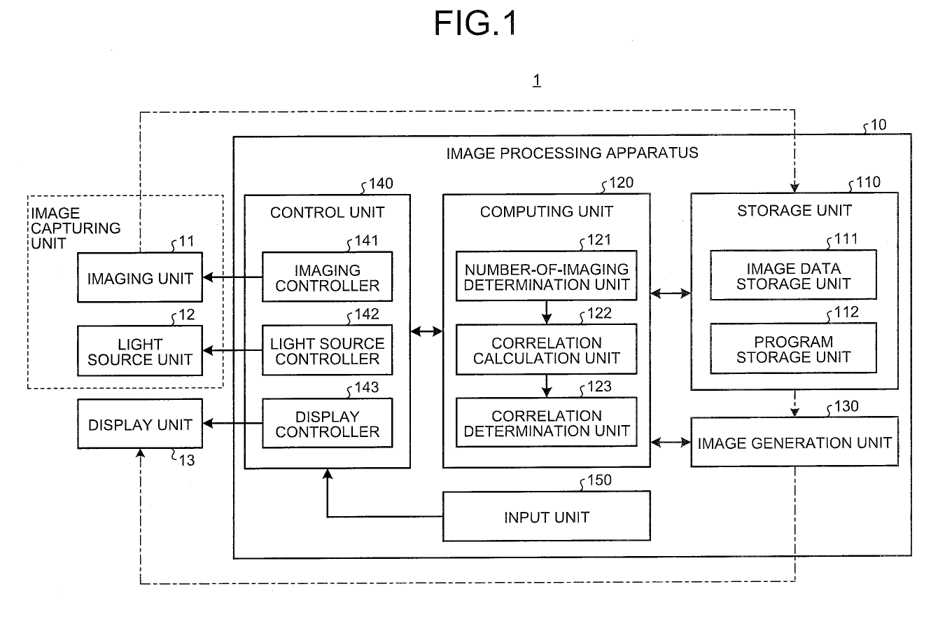

FIG. 1 is a block diagram illustrating an imaging system including an image processing apparatus according to a first embodiment of the present invention. An imaging system 1 illustrated in FIG. 1 irradiates a subject with normal light, performs normal light imaging to generate image data representing a normal light image (first image) based on the normal light (light having first spectral characteristics) reflected from the subject and also special light imaging to generate image data representing a special light image (second image) based on special light (light having second spectral characteristics) having a limited band relative to the normal light, and displays an image based on the image data generated by the respective imaging. The above-described imaging system 1 is applied to, for example, an endoscope system that images inside of a lumen of a living body and displays an image of the inside of the lumen.

The imaging system 1 includes an image processing apparatus 10, an imaging unit 11 adapted to image a subject and generate image data under the control of the image processing apparatus 10, a light source unit 12 adapted to generate light to irradiate the subject under the control of the image processing apparatus 10, and a display unit 13 adapted to display an image applied with image processing by the image processing apparatus 10. Among these units, the imaging unit 11 and the light source unit 12 constitute an image capturing unit that performs normal light imaging and special light imaging.

The imaging unit 11 includes: an image sensor such as CCD adapted to generate and output an imaging signal by photoelectrically converting received light; and an optical system adapted to form a subject image represented by light reflected from the subject on a light receiving surface of the image sensor. The imaging unit 11 performs operation at a preset frame rate under the control of a control unit 140 described later.

The light source unit 12 includes: a simultaneous type white light source such as a white LED or a xenon lam; a filter disposed in an insertable/removable manner on an optical path of white light emitted from the white light source, and functioning as a wavelength selecting unit adapted to transmit special light out of the white light having specific spectral characteristics; and a switching unit adapted to switch the filter between an inserted state and a removed state on the optical path of the white light under the control of the control unit 140.

While the filter is being inserted to the optical path of the white light, the subject is irradiated with the special light, and an image generated by performing imaging during this time is to be a special light image. On the other hand, while the filter is being removed from the optical path of the white light, the subject is irradiated with the normal light, and an image generated by performing imaging during this time is to be a normal light image.

Instead of inserting/removing the filter on the optical path of the white light, a liquid crystal tunable filter, an acousto-optical tunable filter, or the like may be disposed on the optical path, and switching between the normal light, namely, the white light and the special light may be performed by electric control.

The display unit 13 is formed of a display device such as an LCD or an EL display, and displays an image of the subject in a predetermined form under the control of the control unit 140.

The image processing apparatus 10 includes: a storage unit 110 adapted to store image data, various kinds of programs, and the like; a computing unit 120 adapted to perform predetermined arithmetic processing based on the image data stored in the storage unit 110; and an image generation unit 130 adapted to form an image based on the image data stored in the storage unit 110; a control unit 140 adapted to control operation of the entire imaging system 1; and an input unit 150 adapted to input, to the control unit 140, a signal in accordance with to operation of the outside.

The storage unit 110 is formed of various kinds of IC memories such as a RAM or a ROM like a rewritable flash memory, a hard disk incorporated or connected via a data communication terminal, or an information recording device such as a CD-ROM and a reading device therefor, or the like. The storage unit 110 includes an image data storage unit 111 adapted to acquire and store the image data generated by the imaging unit 11, and a program storage unit 112 adapted to store various kinds of programs. Specifically, the program storage unit 112 stores a program that causes the imaging system 1 to perform a series of imaging in which normal light imaging is performed at a preset frame rate and also special light imaging is performed instead of the normal light imaging in the case where a predetermined condition is satisfied.

The computing unit 120 includes a number-of-imaging determination unit 121 adapted to determine whether normal light imaging is consecutively performed a predetermined number of times or more; a correlation calculation unit 122 adapted to calculate a correlation value that is a parameter representing a degree of correlation between a normal light image and a special light image; and a correlation determination unit 123 adapted to determine whether there is correlation between the normal light image and the special light image based on the correlation value.

The image generation unit 130 generates a normal light image and a special light image based on the image data stored in the image data storage unit 111. More specifically, the image generation unit 130 generates an image for display by applying, to the image data stored in the image data storage unit 111, white balance adjustment processing, gain adjustment processing, y correction processing, D/A conversion processing, format change processing, and the like, for example.

The control unit 140 is implemented by hardware such as a CPU, performs transmission of a command and data to the respective units constituting the imaging system 1 in accordance with image data received from the imaging unit 11 and various kinds of signals received from the input unit 150 by reading the various kinds of programs stored in the program storage unit 112, and integrally controls operation of the entire imaging system 1.

More specifically, the control unit 140 includes an imaging controller 141, a light source controller 142, and a display controller 143. The imaging controller 141 causes the imaging unit 11 to perform imaging at the preset frame rate. The light source controller 142 causes the light source unit 12 to generate the normal light to irradiate the subject consecutively or intermittently in synchronization with the frame rate and also generate the special light at specific timing instead of the normal light. The display controller 143 causes the display unit 13 to display the normal light image and the special light image in a predetermined form.

The input unit 150 is formed of input devices such as a keyboard, a touch panel, and various kinds of switches, and outputs, to the control unit 140, input signals generated in accordance with operation made to these input devices from the outside.

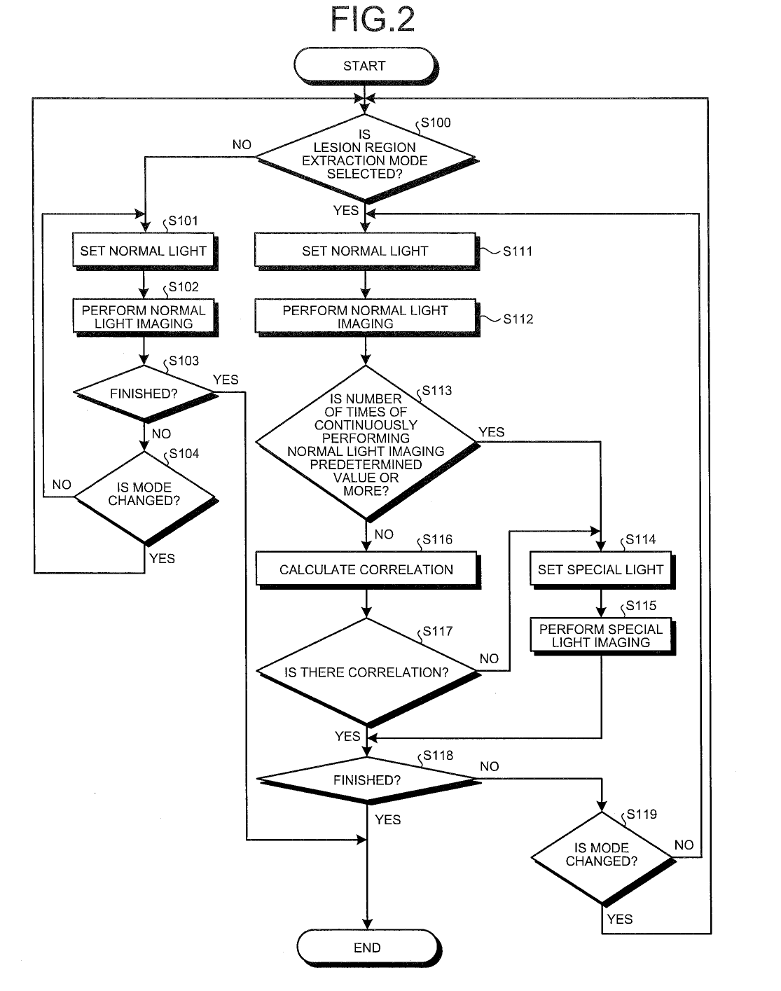

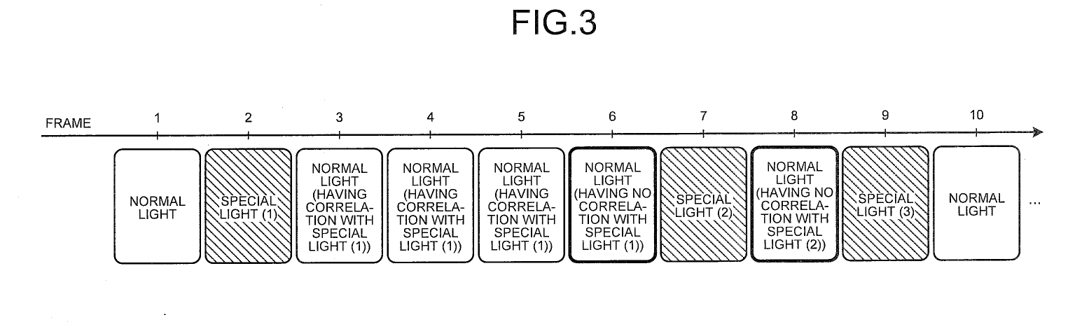

Next, operation of the imaging system 1 will be described. FIG. 2 is a flowchart illustrating operation of the imaging system 1. Additionally, FIG. 3 is a schematic diagram illustrating an image sequence sequentially generated by the imaging system 1. In FIG. 3, special light images (special light (1), (2), (3)) are indicated by hatching.

First, in Step S100, the control unit 140 determines whether a lesion region extraction mode is selected. Here, the lesion region extraction mode means a mode to obtain a special light image in which a specific structure such as a vessel or a tumor is highlighted by performing special light imaging between normal light imaging. The lesion region extraction mode is selected in accordance with predetermined input operation to the input unit 150.

In the case where the lesion region extraction mode is not selected (Step S100: No), the light source controller 142 performs setting so as to cause the light source unit 12 to generate the normal light (Step S101).

In subsequent Step S102, the imaging controller 141 causes the imaging unit 11 to perform imaging. Image data generated by this normal light imaging is received in the image processing apparatus 10 from the imaging unit 11 and stored in the image data storage unit 111. In response to this, the image generation unit 130 reads the image data and generates a normal light image, and outputs the same to the display unit 13. The display unit 13 displays the normal light image under the control of the display controller 143.

In Step S103, the control unit 140 determines whether a signal to command finish is received from the input unit 150. In the case where the signal to command finish is received (Step S103: Yes), the imaging system 1 finishes operation.

On the other hand, in the case where the signal to command finish is not received (Step S103: No), the control unit 140 determines whether a signal to command mode change is received from the input unit 150 (Step S104). In the case where the signal to command mode change is received (Step S104: Yes), operation of the imaging system 1 returns to Step S100. On the other hand, in the case where the signal to command mode change is not received (Step S104: No), operation of the imaging system 1 returns to Step S101.

The control unit 140 causes the each unit of the imaging system 1 to perform the above-described series of Steps S101 to S104 at the preset frame rate. Consequently, normal light images are sequentially displayed in a moving image form on the display unit 13. The frame rate may be a fixed value preliminarily set in the imaging system 1, or a desired value may be set by user's operation using the input unit 150.

In Step S100, in the case where the lesion region extraction mode is selected (Step S100: Yes), the light source controller 142 first performs setting so as to cause the light source unit 12 to generate the normal light (Step S111).

In subsequent Step S112, the imaging controller 141 causes the imaging unit 11 to perform imaging. Image data generated by this normal light imaging is received in the image processing apparatus 10 from the imaging unit 11 and stored in the image data storage unit 111. In response to this, the computing unit 120 counts the number of times of the normal light imaging consecutively performed. Furthermore, the image generation unit 130 reads the image data and generates a normal light image, and outputs the same to the display unit 13. The display unit 13 displays the normal light image under the control of the display controller 143.

In Step S113, the number-of-imaging determination unit 121 determines whether the number of times of continuously performing the normal light imaging is a predetermined value or more. If the number of times of continuously performing the normal light imaging is the predetermined value or more (Step S113: Yes), the light source controller 142 performs setting so as to cause the light source unit 12 to generate the special light (Step S114).

In subsequent Step S115, the imaging controller 141 causes the imaging unit 11 to perform imaging. Image data generated by this special light imaging is received in the image processing apparatus 10 from the imaging unit 11 and stored in the image data storage unit 111. In response to this, the image generation unit 130 reads the image data and generates a special light image. FIG. 3 illustrates a state in that a normal light image is generated in a first frame and subsequently a special light image (special light (1)) is formed in a next second frame. Additionally, the display unit 13 displays the generated special light image under the control of the display controller 143. A display form of the special light image will be described later.

Subsequently in Step S118, the control unit 140 determines whether a signal to command finish is received from the input unit 150. In the case where the signal to command finish is received (Step S118: Yes), the imaging system 1 finishes operation.

On the other hand, in the case where the signal to command finish is not received (Step S118: No), the control unit 140 determines whether a signal to command mode change is received from the input unit 150 (Step S119). In the case where the signal to command mode change is received (Step S119: Yes), operation of the imaging system 1 returns to Step S100. On the other hand, in the case where the signal to command mode change is not received (Step S119: No), operation of the imaging system 1 returns to Step S111.

If the number of times of continuously performing the normal light imaging is less than the predetermined value (Step S113: No), the correlation calculation unit 122 performs correlative calculation between a latest normal light image generated by the normal light imaging in Step S112 and a special light image formed last in this stage, and calculates a correlation value between the both images (Step S116). For example, when a normal light image is formed in a third frame, correlative calculation with the special light image (special light (1)) formed in the second frame is performed.

A method of correlative calculation in Step S116 is not particularly limited, and as far as a parameter representing a degree of correlation can be calculated, known various kinds of methods can be applied. In the first embodiment, calculation in which the stronger correlation is, the larger a correlation value becomes is performed. Specifically, normalized cross-correlation (NCC) by template matching is calculated as the correlation value. According to the NCC, the stronger correlation between images is, the larger the value becomes.

In Step S117, the correlation determination unit 123 determines whether there is correlation between the images to be determined. In the first embodiment, in the case where the correlation value calculated in Step S116 is a threshold or more, it is determined that there is correlation between the images to be determined.

If it is determined that there is correlation between the images to be determined (Step S117: Yes), it can be considered that change is little in a visual field of the imaging unit 11 from a frame in which special light imaging is performed previously. In this case, operation of the imaging system 1 proceeds to Step S118, and the normal light imaging is repeated (refer to Step S111) when no command to finish imaging or no command for mode change is received (refer to Step S118, S119). For example, in the case where the normal light image is formed in the third frame, when it is determined that there is correlation with the special light image (special light (1)) formed in the second frame, the normal light imaging is performed in a next fourth frame.

In contrast, in the case where it is determined that there is no correlation between the images to be determined (Step S117: No), it can be considered that change is large in the visual field of the imaging unit 11 from the frame in which special light imaging is performed previously. In this case, operation of the imaging system 1 proceeds to Step S114, and performs the special light imaging. For example, in the case where the normal light image is formed in a sixth frame, when it is determined that there is no correlation with the special light image (special light (1)) formed in the second frame, the special light imaging is performed in a next seventh frame.

Thus, in the lesion region extraction mode, the special light imaging is performed between the normal light imaging in accordance with determination results in Step S113 and S117. As a result, as illustrated in FIG. 3, the normal light image or the special light image is obtained at the preset frame rate.

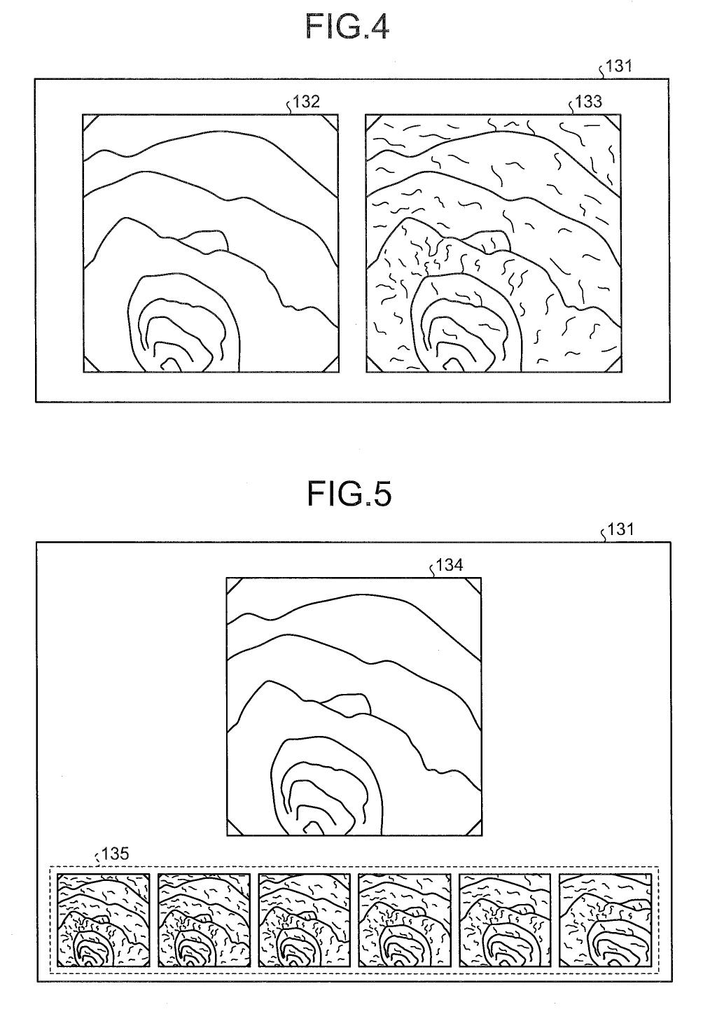

FIG. 4 is a schematic diagram illustrating exemplary display of a normal light image and a special light image in the lesion region extraction mode. As illustrated in FIG. 4, a normal light image display area 132 and a special light image display area 133 are provided on a screen 131 of the display unit 13. In the normal light image display area 132, the normal light image formed in Step S112 is displayed in a moving image form. On the other hand, in the special light image display area 133, the special light image formed in Step S115 is displayed in a sequentially switched manner.

FIG. 5 is a schematic diagram illustrating different exemplary display of the normal light image and the special light image in the lesion region extraction mode. As illustrated in FIG. 5, a normal light image display area 134 and a thumbnail area 135 are provided on the screen 131 of the display unit 13. In the normal light image display area 134, the normal light image formed in Step S112 is displayed in a moving image form. On the other hand, in the thumbnail area 135, the special light image formed in Step S115 is reduced in size and displayed as a still image in a listed manner. In FIG. 5, an example of arranging reduced images of special light images in a line below the normal light image display area 134, but arrangement of the reduced images is not limited thereto, and for example, the reduced images may also be arranged in a manner surrounding the normal light image display area 134.

As described above, according to the first embodiment of the present invention, when the normal light imaging is performed at the preset frame rate, and when the normal light imaging is consecutively performed the predetermined number of times or more and when there is no correlation between the normal light image and the special light image, the special light imaging is performed instead of the normal light imaging. Therefore, decrease of the frame rate of the normal light imaging can be suppressed, and a moving image can be played back with high image quality, and furthermore, the special light image can be formed without omission at necessary timing such as when there is significant change in the visual field. Moreover, according to the first embodiment, the special light image is displayed on the screen together with the normal light image. Therefore, a user can observe a feature region highlighted in the special light image while referring to the normal light image.

### Modified Example 1-1

Next, a modified example 1-1 of the first embodiment of the present invention will be described. In the above-described first embodiment, the correlation calculation unit 122 calculates the NCC as the correlation value between the normal light image and the special light image, but a known parameter may also be calculated as the correlation value other than this. Specifically, a sum of squared difference (SSD) and a sum of absolute difference (SAD) may be exemplified. According to the SSD and the SAD, the stronger correlation between images is, the smaller a value becomes. Therefore, in this case, when the SSD or the SAD is larger than a threshold, it is determined in Step S117 that there is no correlation, and the special light imaging is performed in a next frame (refer to Steps S114 and S115). In contrast, in this case, when the SSD or the SAD is smaller than the threshold, it is determined that there is correlation, and the normal light imaging is performed in a next frame (refer to Steps S111 and S112).

Alternatively, a corresponding point between the two images is extracted and a moving amount of the corresponding point of these images may be calculated as a parameter representing correlation between the normal light image and the special light image. In this case, when the moving amount is larger than a threshold, it is determined that there is no correlation, and the special light imaging is performed in a next frame. In contrast, when the moving amount is less than the threshold, it is determined that there is correlation and normal light imaging is performed in a next frame.

Also, the corresponding point between the two images is extracted and a change amount of luminance or a color at the corresponding point of these images may also be calculated as the parameter representing correlation between the normal light image and the special light image. In this case, when the change amount is larger than a threshold, it is determined that there is no correlation, and the special light imaging is performed in a next frame. In contrast, when the change amount is less than the threshold, it is determined that there is correlation and normal light imaging is performed in a next frame.

### Modified Example 1-2

Next, a modified example 1-2 of the first embodiment of the present invention will be described. In the first embodiment, as the structure of the light source unit 12, the normal light and the special light are switched by inserting/removing the filter on the optical path of the white light emitted from the light source, but the structure of the light source unit 12 is not limited thereto. For example, the normal light and the special light may also be switched by providing a white light source that emits white light and a special light source such as an LED which emits the special light, and connecting, to any one of these white light source and special light source, the optical path up to an emission port of light to irradiate a subject.

### Modified Example 1-3

Next, a modified example 1-3 of the first embodiment of the present invention will be described. In the above-described first embodiment, the timing to perform the special light imaging instead of the normal light imaging is controlled based on the number of times of consecutive execution of the normal light imaging, and the correlation between the latest normal light image and the special light image formed last. However, in addition to this, there may be a structure in which the special light imaging is performed at the timing desired by a user.

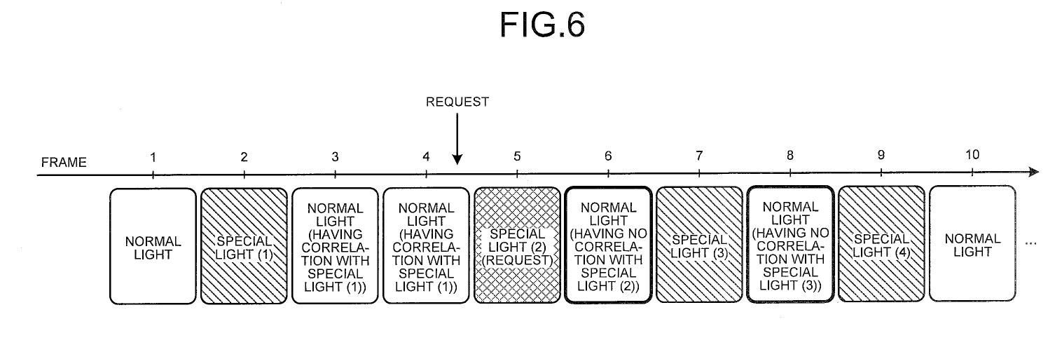

FIG. 6 is a schematic diagram illustrating an image sequence sequentially formed in the modified example 1-3. Note that special light images (special light (1), (2), (3), (4)) are indicated by hatching in FIG. 6.

In the modified example 1-3 also, as in the first embodiment, the special light imaging is basically performed when normal light imaging is consecutively performed a predetermined number of times and when there is no correlation with between the latest normal light image and the special light image formed last. For example, as illustrated in FIG. 6, in the case where a normal light image is formed in a third frame, correlation between this normal light image and the special light image (special light (1)) formed in the second frame is determined. Then, when it is determined that there is correlation, the normal light imaging is performed in a next fourth frame.

In the case where a request signal to perform the special light imaging is received from the input unit 150 during execution of the normal light imaging, the special light imaging is performed in a next frame. For example, when the request signal is received during execution of the normal light imaging in the fourth frame, the special light imaging is performed in a next fifth frame. In this case, the normal light image is further performed in a next sixth frame, and determination is made on correlation between the normal light image generated by this normal light imaging and the special light image (special light (2)) formed last in this stage by the request.

### Modified Example 1-4

Next, a modified example 1-4 of the first embodiment of the present invention will be described. In the above-described first embodiment, the normal light imaging and the special light imaging are switched by controlling spectral characteristics of light to irradiate the subject, but spectral characteristics of light that is reflected from the subject and enters an image sensor may also be controlled.

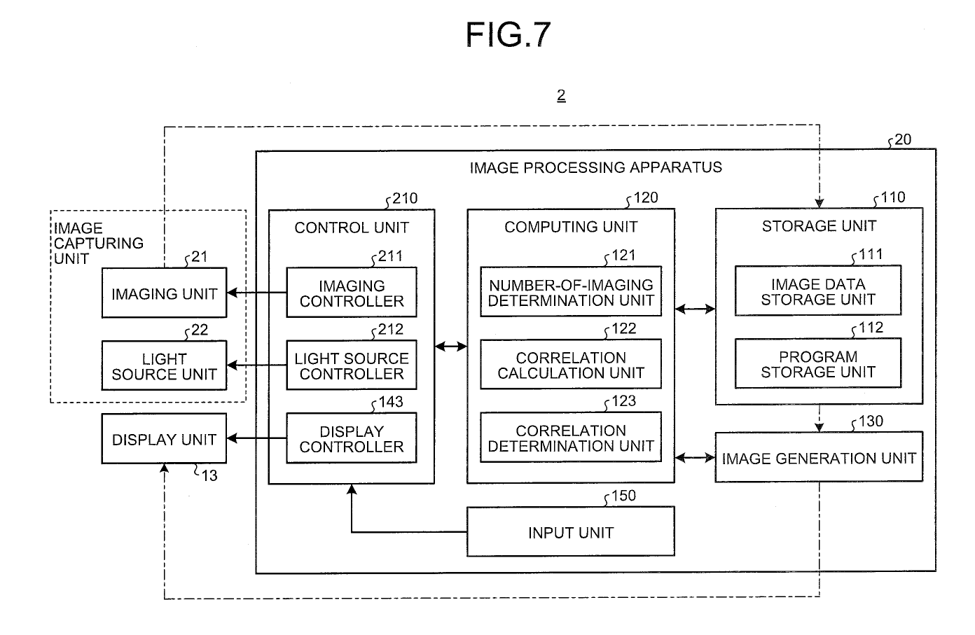

FIG. 7 is a block diagram illustrating a configuration of an imaging system according to the modified example 1-4. As illustrated in FIG. 7, an imaging system 2 according to the modified example 1-4 includes an image processing apparatus 20, an imaging unit 21, a light source unit 22, and a display unit 13. Among them, the imaging unit 21 and the light source unit 22 constitute an image capturing unit. Furthermore, a configuration and operation of the display unit 13 are the same as the first embodiment.

The imaging unit 21 includes: an image sensor such as a CCD or a CMOS adapted to generate and output an imaging signal by photoelectrically converting received light; a filter disposed in an insertable/removable manner on an optical path of light incident to the image sensor, and functioning a wavelength selecting unit adapted to transmit a component (special light) having specific spectral characteristics; and a switching unit adapted to switch the filter between an inserted state and a removed state on the optical path of the incident light to the image sensor under the control of a control unit 210.

The light source unit 22 is a light source that generates white light also called as normal light, and performs operation under the control of the control unit 210, and irradiates a subject with the normal light.

While the filter is being inserted to the optical path of the incident light, a special light component included in the normal light reflected from the subject enters the image sensor, and an image generated by performing imaging during this time is to be a special light image. On the other hand, while the filter is being removed from the optical path of the incident light, the normal light reflected from the subject enters the image sensor, and an image generated by performing imaging during this time is to be a normal light image.

The image processing apparatus 20 includes, instead of the control unit 140 illustrated in FIG. 1, the control unit 210 having an imaging controller 211, a light source controller 212, and a display controller 143. The imaging controller 211 causes the imaging unit 21 to perform imaging at a preset frame rate and further controls the switching unit included in the imaging unit 21. Consequently, imaging is switched between the normal light imaging in which the normal light is received and image data representing a normal light image is generated and the special light imaging in which the special light is received and image data representing a special light image is generated. The light source controller 212 controls generating operation of the normal light by the light source unit 22. Operation of the display controller 143 is the same as the first embodiment.

### Modified Example 1-5

Next, a modified example 1-5 of the first embodiment of the present invention will be described. In the above-described modified examples 1-4, various kinds of structures other than the filter and the switching unit may be applied as the unit to switch the light between the light entering the image sensor, namely, the normal light and the special light. For example, a wavelength selecting unit such as a liquid crystal tunable filter or an acousto-optical tunable filter (AOTF) may be installed on the optical path of the light entering the image sensor, and an optical characteristic of the light entering the image sensor may be controlled by electric control.

### Second Embodiment

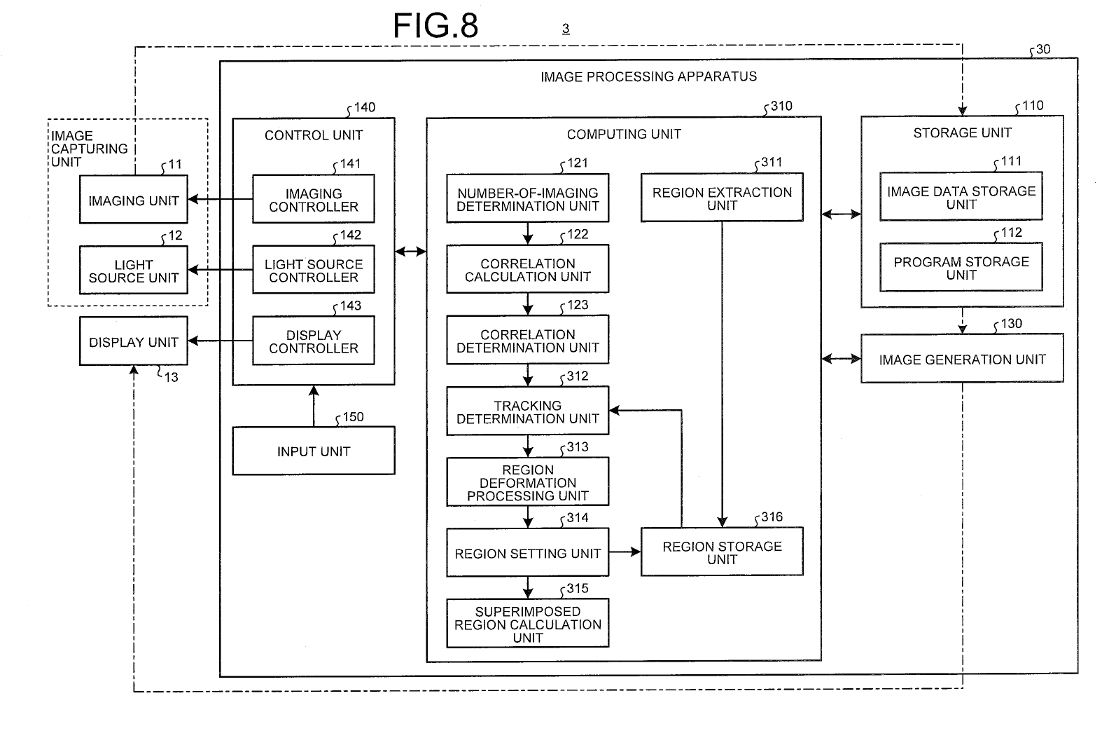

Next, a second embodiment of the present invention will be described. FIG. 8 is a block diagram illustrating a configuration of an imaging system including an image processing apparatus according to the second embodiment of the present invention. As illustrated in FIG. 8, an imaging system 3 according to the second embodiment includes an image processing apparatus 30 instead of an image processing apparatus 10 illustrated in FIG. 1. Configurations of an imaging unit 11, a light source unit 12, and a display unit 13 are the same as the first embodiment (refer to FIG. 1). Alternatively, an imaging unit 21, a light source unit 22, and the display unit 13 may also be provided in the same manner as a modified example 1-4 (refer to FIG. 7).

The image processing apparatus 30 includes a computing unit 310 instead of a computing unit 120 illustrated in FIG. 1. In addition to an number-of-imaging determination unit 121 to a correlation determination unit 123, the computing unit 310 includes: a region extraction unit 311 adapted to extract, as a region of interest, a feature region such as a lesion from a special light image; a tracking determination unit 312 adapted to determine whether the region of interest can be tracked in a latest normal light image; a region deformation processing unit 313 adapted to deform a shape of the region of interest in accordance with a determination result of the tracking determination unit 312; a region setting unit 314 adapted to set the deformed region of interest as a latest region of interest; a superimposed region calculation unit 315 adapted to calculate a region in which the latest region of interest is displayed in a manner superimposed on the normal light image; and a region storage unit 316 adapted to store the latest region of interest. Operation in the number-of-imaging determination unit 121 to the correlation determination unit 123 is the same as the first embodiment. Additionally, a configuration and operation of the image processing apparatus 30 other than the computing unit 310 are the same as the first embodiment.

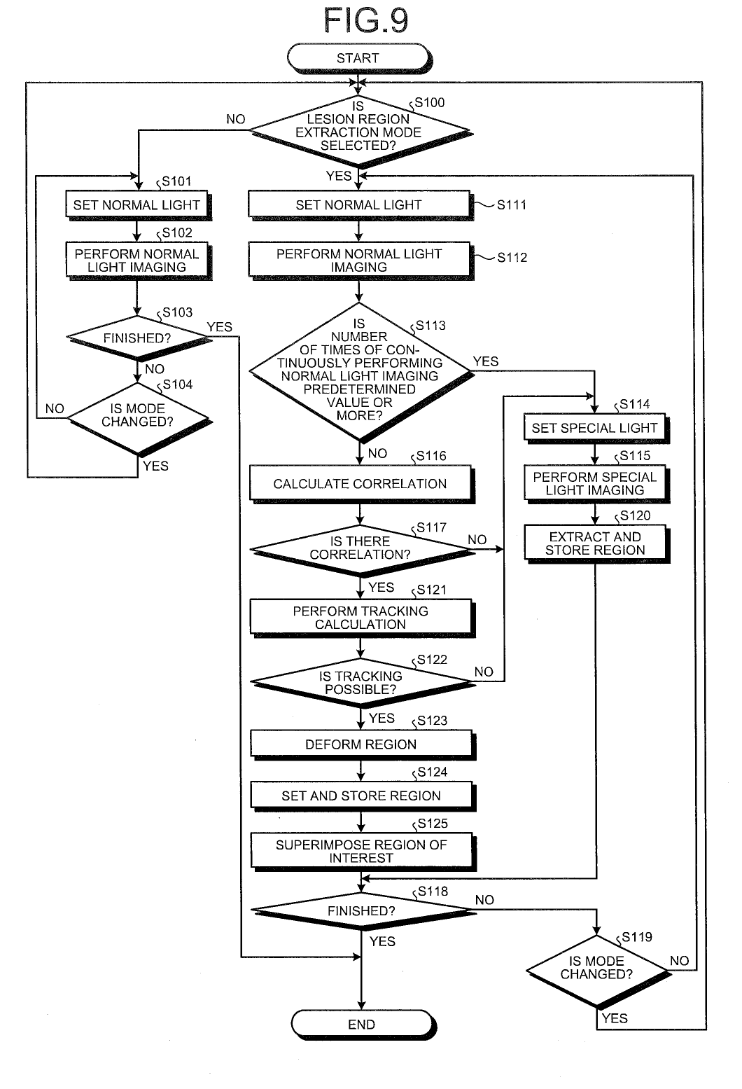

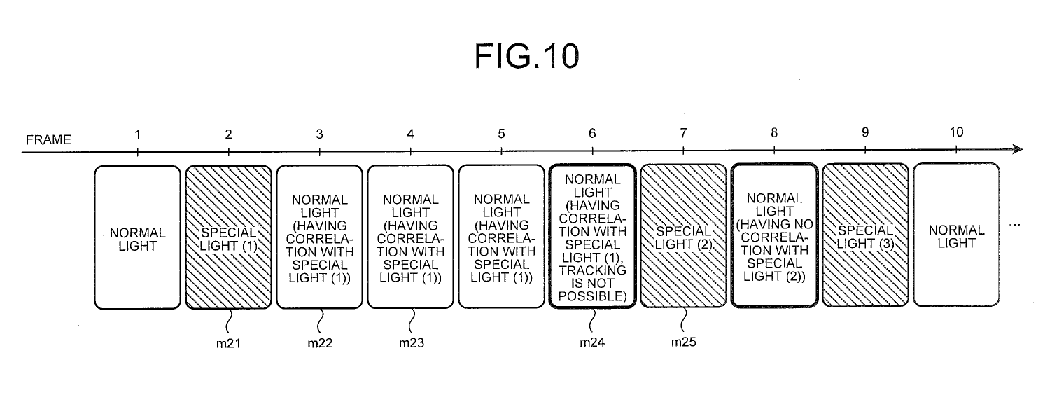

Next, operation of the imaging system 3 will be described. FIG. 9 is a flowchart illustrating operation of the imaging system 3. Additionally, FIG. 10 is a schematic diagram illustrating an image sequence sequentially generated by the imaging system 3. In FIG. 10, special light images (special light (1), (2), (3)) are indicated by hatching.

Operation in Steps S100 to S104, S111 to S115, S116, and S117 illustrated in FIG. 9 are the same as the first embodiment.

In Step S120 subsequent to Step S115, the region extraction unit 311 extracts, as a region of interest, a feature region such as a lesion from a special light image based on image data of a special light image stored in an image data storage unit 111 by a known method such as performing threshold processing for a pixel value, and then causes the region storage unit 316 to update and store the extracted region of interest as a latest region of interest. After that, operation of the imaging system 3 proceeds to Step S118. Operation in Steps S118 and S119 is the same as the first embodiment.

Further, in Step S117, in the case where it is determined that there is correlation between a normal light image and a special light image (Step S117: Yes), the tracking determination unit 312 performs tracking calculation for the normal light image formed in Step S112 relative to the region of interest stored in the region storage unit 316 (Step S121). As the tracking calculation, for example, a method such as template matching may be applied.

In subsequent Step S122, the tracking determination unit 312 determines whether the region of interest can be tracked in the normal light image based on a result of the tracking calculation in Step S121. In the case where it is determined that the region of interest can be tracked (Step S122: Yes), the region deformation processing unit 313 deforms the region of interest to conform to a shape of a corresponding region inside the normal light image (Step S123).

In Step S124, the region setting unit 314 sets, as a latest region of interest, the region of interest deformed in Step S123, and causes the region storage unit 316 to update and store the same.

In Step S125, the superimposed region calculation unit 315 calculates a region inside the latest normal light image corresponding to the region of interest stored in the region storage unit 316, and superimposes the region of interest on the normal light image at the same position of the region, and displays the superimposed image. Superimposing of the region of interest on the normal light image will be described later. Operation in subsequent Steps S118 and S119 is the same as the first embodiment.

For example, in the case where a normal light image m22 is formed in a third frame (refer to Step S112), when there is correlation between the normal light image m22 and a special light image (special light (1)) m21 formed last at this stage and also a region of interest extracted from the special light image m21 can be tracked in the normal light image m22, the region of interest extracted from the special light image m21 is deformed to conform to a shape of a corresponding region inside the normal light image m22 and the region of interest as deformed is stored in the region storage unit 316. Then, the deformed region of interest is superimposed on the normal light image m22.

Additionally, in the case where a normal light image m23 is formed in a fourth frame (refer to Step S112), when there is correlation between the normal light image m23 and a special light image (special light (1)) m21 formed last at this stage and also a region of interest stored in the region storage unit 316 can be tracked in the normal light image m23, the region of interest is further deformed to conform to a shape of a corresponding region inside the normal light image m23 and the region of interest as deformed is stored in the region storage unit 316. Then, the deformed region of interest is superimposed on the normal light image m23.

On the other hand, in the case where it is determined in Step S122 that the region of interest cannot be tracked in the normal light image (Step S122: No), it is necessary to newly set a region of interest. Therefore, operation of the imaging system 3 proceeds to Step S114, and performs the special light imaging.

For example, in the case where a normal light image m24 is formed in a sixth frame (refer to Step S112), when there is correlation between the normal light image m24 and the special light image (special light (1)) m21 formed last at this stage but the region of interest stored in the region storage unit 316 cannot be tracked in the normal light image m24, the special light imaging is performed in a next seventh frame. In this case, a region of interest extracted from a special light image m25 is updated and stored in the region storage unit 316.

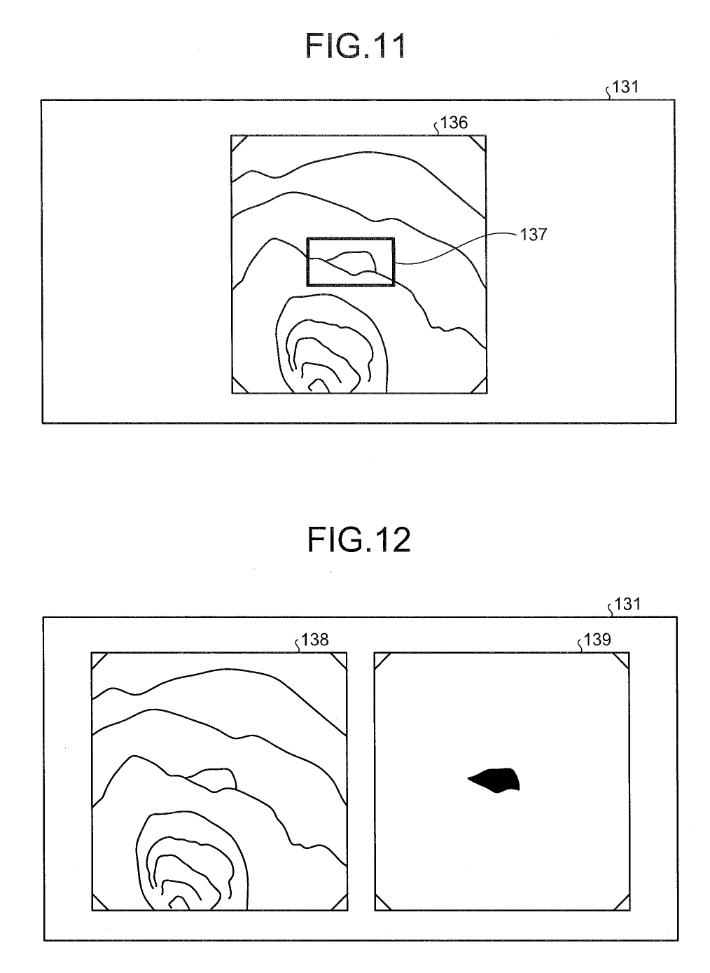

FIG. 11 is a schematic diagram illustrating exemplary display of a normal light image and a special light image in a lesion region extraction mode. As illustrated in FIG. 11, an image display area 136 is provided on a screen 131 of the display unit 13. In the image display area 136, the normal light image formed in Step S112 is displayed in a moving image form, and also a frame 137 surrounding a region corresponding to the region of interest stored in the region storage unit 316 is displayed in a superimposed manner. Alternatively, instead of the frame 137, for example, highlighting by increasing luminance of the region inside the normal light image corresponding to the region of interest, coloring the region with a specific color, or surrounding a contour of the region may also be performed. Furthermore, an image of the region of interest stored in the region storage unit 316 may also be displayed in a manner superimposed on the normal light image. Thus, by displaying the normal light image and the region of interest in an associated manner, a user can instantly grasp a region to be intensively observed.

As in the first embodiment, in the above-described image display area 136, the special light image may be displayed next thereto (refer to FIG. 4) or reduced images of special light images may be displayed in a line as thumbnails (refer to FIG. 5). Additionally, in the case where it is determined in Step S117 that there is no correlation between images to be determined, highlighting such as the frame 137 may be erased.

FIG. 12 is a schematic diagram illustrating different exemplary display of the normal light image and the special light image in the lesion region extraction mode. As illustrated in FIG. 12, a normal light image display area 138 and a region of interest display area 139 are provided on the screen 131 of the display unit 13. In the normal light image display area 138, the normal light image formed in Step S112 is displayed in a moving image form. On the other hand, in the region of interest display area 139, the region of interest stored in the region storage unit 316 is sequentially updated and displayed. Alternatively, in the region of interest display area 139, highlighting such as displaying only a contour of the region of interest, coloring the region of interest with a specific color, or the like may also be performed.

As described above, according to the second embodiment of the present invention, when normal light imaging is performed at a preset frame rate and the normal light imaging is consecutively performed a predetermined number of times or more, and when there is no correlation between a latest normal light image and a special light image formed last or when the region of interest cannot be tracked in the latest normal light image, the special light imaging is performed instead of the normal light imaging. Therefore, decrease of the frame rate can be suppressed in the normal light imaging, and a moving image can be played back with high image quality, and furthermore, the special light image can be formed without omission at necessary timing such as when there is significant change in a visual field and when a specific region such as a lesion enters a range of view. Moreover, according to the second embodiment, the region of interest extracted from the special light image is deformed to conform to the corresponding region inside the normal light image. Therefore, the region of interest can be properly superimposed on the normal light image, and the user can correctly grasp a position of the region of interest in the normal light image.

### Third Embodiment

Next, a third embodiment of the present invention will be described. A control method for timing to perform special light imaging is not limited to first and second embodiments, and the timing can be controlled by various kinds of methods. For example, an execution ratio between normal light imaging and special light imaging may be fixed, and furthermore, the special light imaging may be performed as needed based on a user's command. A configuration of an imaging system according to the third embodiment is the same as the second embodiment (refer to FIG. 8).

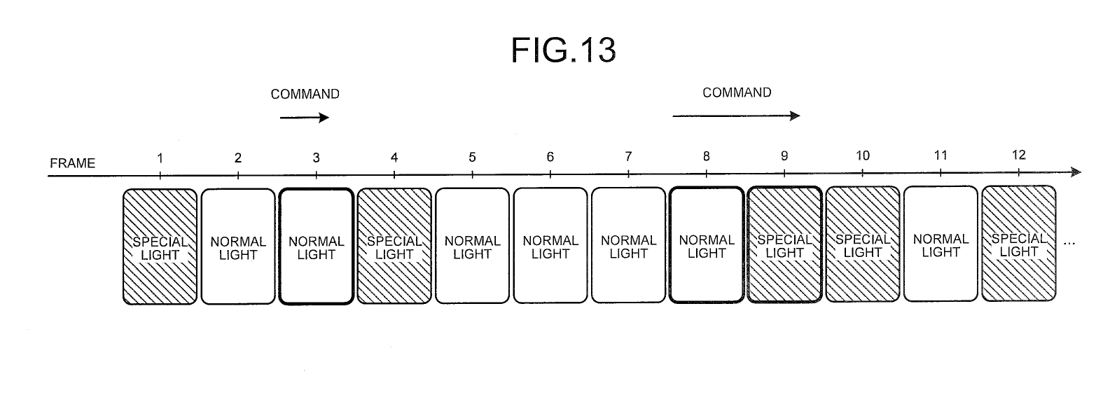

FIG. 13 is a schematic diagram illustrating an image sequence sequentially formed in a third embodiment of the present invention. In FIG. 13, a special light image is indicated by hatching. Furthermore, in the third embodiment, setting is made such that the special light imaging is performed every time normal light imaging is performed ten times.

In this case, basically, the normal light imaging is consecutively performed ten times after a special light image is formed in a first frame by special light imaging, and a special light image is generated by executing the special light imaging again in a later twelfth frame. During this time, in the case where a command signal to command execution of the special light imaging is received from an input unit 150, a control unit 140 causes an imaging unit 11 and a light source unit 12 to perform the special light imaging in a next frame of the timing at which the command signal is received. For example, in a case of FIG. 13, since the command signal is received during a third frame, the special light imaging is performed in a next fourth frame. Also, since the command signal is consecutively received in eighth and ninth frames, the special light imaging is performed in ninth and tenth frames. In the twelfth frame, the special light imaging is performed as originally scheduled.

When the special light imaging is performed in the third embodiment, a computing unit 310 may extract a region of interest from the special light image same as the second embodiment, then may deform the region of interest to conform to a corresponding region inside a latest normal light image, and may update and store the same in the region storage unit 316. In this case, the display unit 13 displays the region of interest, or a frame or a mark indicating the region of interest in a manner superimposed on the normal light image. Alternatively, as in the first embodiment, the normal light image and the special light image may be simply displayed side by side without extracting the region of interest.

According to the third embodiment of the present invention, decrease of a frame rate of the normal light imaging can be suppressed, and a moving image can be played back with high image quality by reducing the execution ratio of special light imaging relative to the normal light imaging. Furthermore, since the special light imaging is performed as needed in accordance with the user's command, a special light image updated in accordance with necessity can be displayed next to a normal light image, or a region of interest extracted from such a special light image can be displayed in a manner superimposed on the normal light image. Therefore, the user can observe the region of interest highlighted in the special light image while referring to the normal light image.

### Fourth Embodiment

Next, a fourth embodiment of the present invention will be described. In first to third embodiments described above, special light imaging is performed by using one kind of special light, but the special light imaging may also be performed by respectively using plural kinds of special light having spectral characteristics different from normal light and also different from each other.

In the case of performing the special light imaging by using the plural kinds of special light, imaging may be performed in a light source unit 12 illustrated in FIG. 1 by sequentially inserting plural kinds of filters having spectral characteristics different from each other to an optical path of light emitted from a light source. Alternatively, imaging may also be performed by providing the light source unit 12 with plural kinds of LED light sources adapted to emit light having spectral characteristics different from each other and sequentially operating these light sources. Alternatively, in an imaging unit 21 illustrated in FIG. 7, imaging may also be performed by sequentially inserting the plural kinds of filters having the spectral characteristics different from each other to an optical path of light incident to an image sensor. Furthermore, instead of the plural kinds of filters, a wavelength selecting unit in which an optical characteristic is changed by electric control may also be inserted to the optical path.

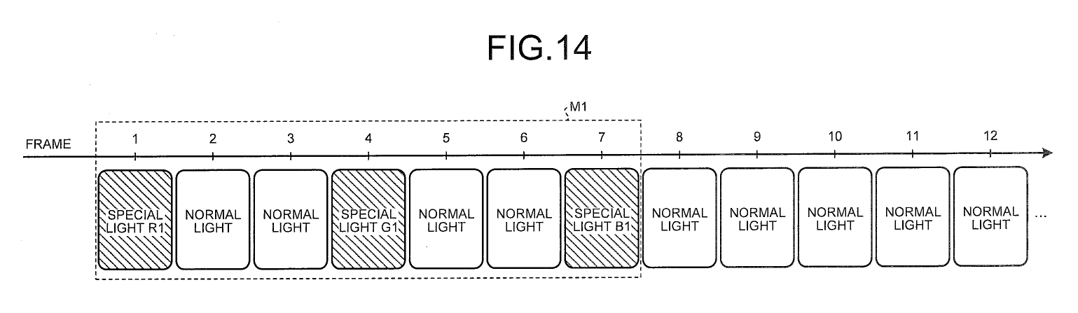

FIG. 14 is a schematic diagram illustrating an image sequence sequentially formed in the fourth embodiment of the present invention. In FIG. 14, a special light image is indicated by hatching.

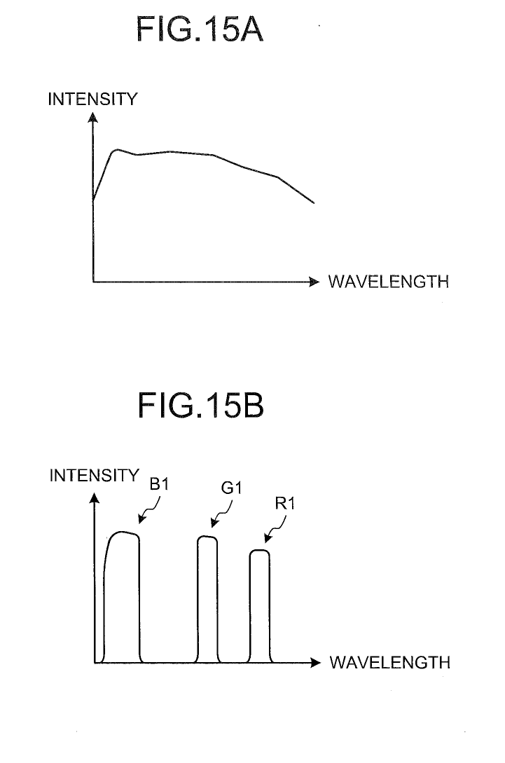

FIGS. 15A and 15B are graphs illustrating exemplary spectral characteristics of light used in imaging in the fourth embodiment. FIG. 15A indicates spectral characteristics (wavelength band) of normal light, and FIG. 15B indicates spectral characteristics (wavelength band) of special light (special light R1, G1, and B1).

Thus, when executing the special light imaging by using the plurality of kinds of special light, frequencies of the special light imaging using the special light R1, G1, and B1 are preferably made equal. Therefore, in the fourth embodiment, imaging is performed, including a set of eight imaging operations in which a predetermined number of times of the normal light imaging operations are inserted between the special light imaging operations using the special light R1, G1 and B1. In FIG. 14, one set of imaging operations is denoted by M1. In FIG. 14, two normal light imaging operations are inserted between the special light imaging operations. Such an imaging set M1 is performed instead of the single special light imaging operation described in the first to third embodiments. More specifically, after the imaging set M1 is performed, the imaging set M1 is performed again when the normal light imaging is continuously performed a predetermined number of times, when there is no correlation between a latest normal light image and a special light image, or when a region of interest stored in the region storage unit 316 cannot be tracked in the latest normal light image although there is the correlation.

Here, in the case of performing correlation determination with the special light image, determination is made on respective correlation with an image of the special light R1, an image of the special light G1, and an image of the special light B1 obtained by the imaging set M1 performed last in a forming stage of the latest normal light image. Then, in the case where there is correlation with any one of these three images, it is determined that there is correlation between the latest normal light image and the imaging set M1.

Furthermore, in the case of performing tracking determination for a region of interest, region of interests extracted from the respective images of the special light R1, G1, B1 are stored in the region storage unit 316 for each of the spectral characteristics of the special light. Then, in the case where any one of the region of interests stored in the region storage unit 316 can be tracked in the latest normal light image, it is determined that the region of interest extracted from the imaging set M1 can be tracked.

According to the fourth embodiment of the present invention, even in the case of using the plural kinds of special light having the spectral characteristics different from each other, an execution ratio of the special light imaging relative to the normal light imaging can be reduced, decrease of the frame rate of the normal light imaging can be suppressed, and a moving image can be played back with high image quality. Additionally, by using the plural kinds of special light having the spectral characteristics different from each other, a region of interest according to spectral characteristics can be extracted and displayed.

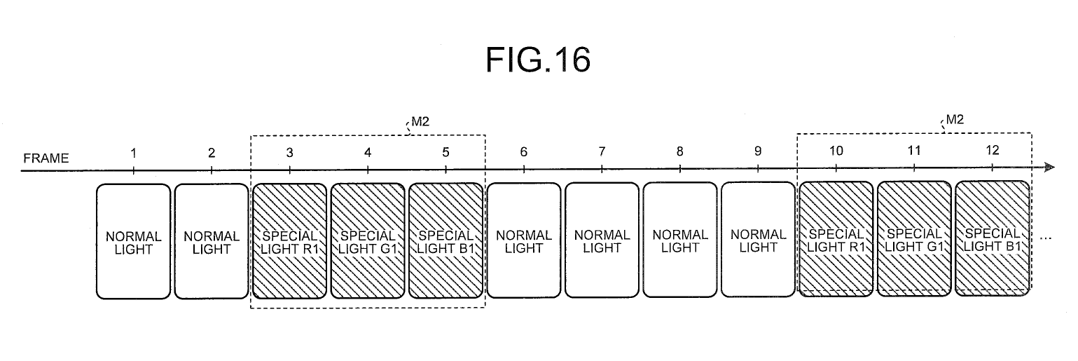

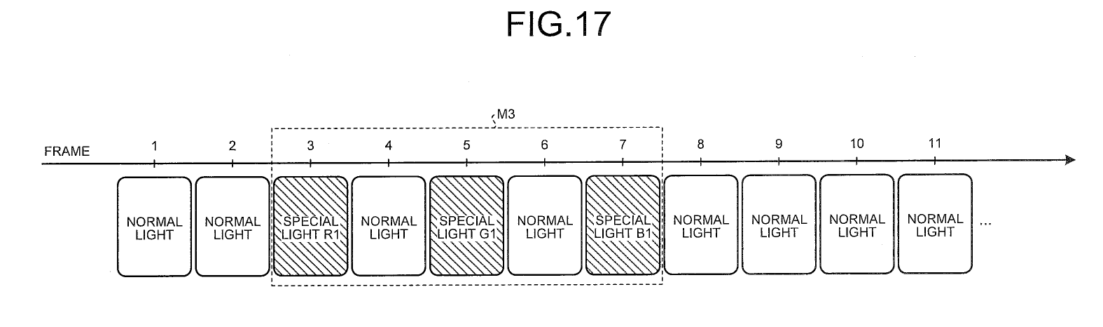

The set of operation to perform imaging by using the plural kinds of special light R1, G1, B1 is not limited to the imaging set M1 illustrated in FIG. 14. For example, like an imaging set M2 illustrated in FIG. 16, imaging may be consecutively performed by using respective special light R1, G1, B1, or like an imaging set M3 illustrated in FIG. 17, imaging by using the respective special light R1, G1, B1 and imaging by using normal light may also be performed alternately. Additionally, kinds of special light used in one set of operation is not limited to three kinds, and may also be two kinds or may also be four or more kinds.

### Fifth Embodiment

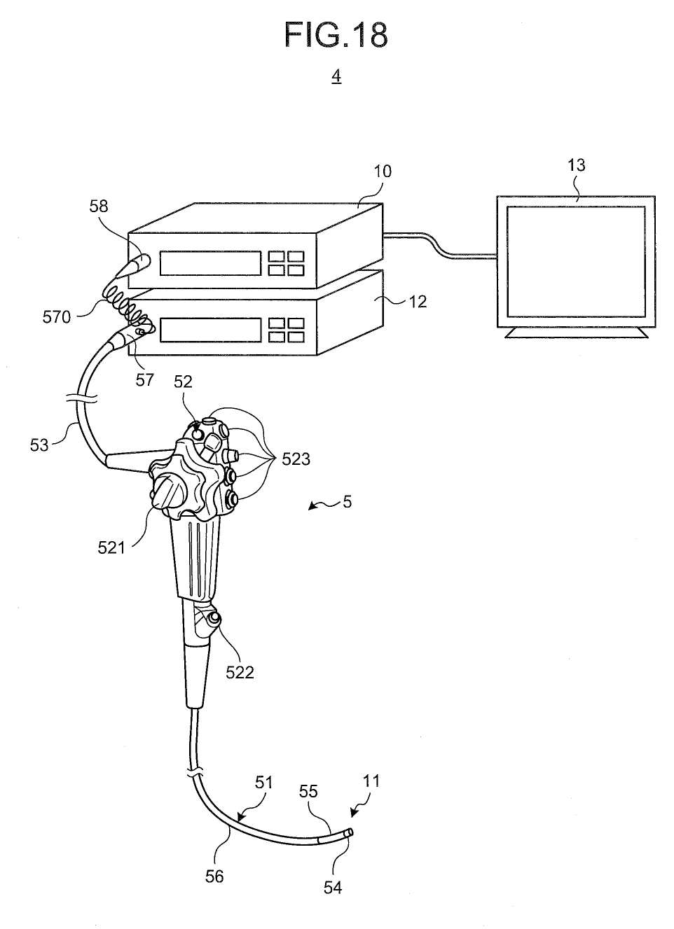

Next, a fifth embodiment of the present invention will be described. FIG. 18 is a schematic diagram illustrating an outline structure of an endoscope system according to the fifth embodiment of the present invention. An endoscope system 4 illustrated in FIG. 18 is one aspect of an imaging system 1 illustrated in FIG. 1 and includes: an image processing apparatus 10; an endoscope 5 adapted to form an image that images inside of a body of a subject by inserting a distal end portion thereof into a lumen of the subject; a light source unit 12 adapted to generate illumination light emitted from a distal end of the endoscope 5; and a display unit 13 adapted to display an in-vivo image applied with image processing by the image processing apparatus 10. The image processing apparatus 10 performs predetermined image processing on the image generated by the endoscope 5 and also integrally controls operation of the entire endoscope system 4. Instead of an image processing apparatus 10 according to a first embodiment, an image processing apparatus 20 according to a modified example 1-4 or an image processing apparatus 30 according to a second embodiment may also be applied.

The endoscope 5 includes: an inserting portion 51 having flexibility and formed in a thin long shape; an operating unit 52 connected to a proximal end side of the inserting portion 51 and adapted to receive input of various kinds of operation signals; and a universal cord 53 extending from the operating unit 52 in a direction different from an extending direction of the inserting portion 51, and incorporating various kinds of cables adapted to connect the image processing apparatus 10 and the light source unit 12.

The inserting portion 51 includes: a distal end portion 54; a bending portion 55 formed of a plurality of bending pieces and capable of being freely bent; and a flexible tube portion 56 connected to a proximal end side of the bending portion 55, having flexibility, and formed in a long shape. An imaging unit 11 is provided at the distal end portion 54 of the inserting portion 51 (refer to FIG. 1).

A cable assembly in which a plurality of signal lines to receive and transmit electric signals with the image processing apparatus 10 is bundled is connected between the operating unit 52 and the distal end portion 54. The plurality of signal lines includes a signal line to transfer a video signal output from an image sensor to the image processing apparatus 10, a signal line to transmit a control signal output from the image processing apparatus 10 to an image sensor, and the like.

The operating unit 52 includes: a bending knob 521 adapted to bend the bending portion 55 in a vertical direction and a horizontal direction; a treatment tool inserting portion 522 adapted to insert a treatment tool such as a living body forceps, a laser scalpel or a test probe; and a plurality of switches 523, namely, an operation input unit adapted to input operation command signals for peripheral apparatuses such as an air feeding unit, a water feeding unit, and a gas feeding unit in addition to the image processing apparatus 10 and the light source unit 12.

The universal cord 53 incorporates at least a light guide and the assembly cable. Furthermore, an end of the universal cord 53 located on a side different from a side connected to the operating unit 52 is provided with: a connector portion 57 detachable to the light source unit 12; and an electric connector portion 58 electrically connected to the connector portion 57 via a coil cable 570 having a coil shape and detachable from the image processing apparatus 10.

The image processing apparatus 10 generates an image to be displayed by the display unit 13 based on image data output from the imaging unit 11 provided at the distal end portion 54. The light source unit 12 generates normal light and special light at predetermined timing under the control of a light source controller 142. The light generated by the light source unit 12 is emitted from a distal end of the distal end portion 54 via the light guide.

In the above-described fifth embodiment, an example of applying the imaging system illustrated in FIG. 1 to the endoscope system for a living body has been described, but the imaging system may also be applied to an endoscope system for industrial use. Alternatively, the imaging system may also be applied to a capsule endoscope introduced into a living body and adapted to perform imaging while moving inside the living body.

In the above-described first to fifth embodiments, the normal light is generated by a white light source of simultaneous lighting, but the normal light may also be generated by a light source of sequential lighting.

According to some embodiments, first image data representing a first image is generated at a preset frame rate based on so-called normal light that is light having first spectral characteristics, and furthermore, timing to generate, instead of the first image data, second image data representing a second image based on so-called special light that is light having second spectral characteristics is controlled based on a determination result of a degree of correlation between the first image and the second image. Consequently, the second image can be suitably generated without largely decreasing an imaging frame rate of the first image. Therefore, the second image can be obtained at necessary timing without omission while preventing degradation of image quality at the time of playing back the first image.

The above-described present invention is not limited to the first to fifth embodiments and the modified examples, and various kinds of invention may also be formed by suitably combining a plurality of elements disclosed in the respective first to fifth embodiments and the modified examples. For example, formation without some of the elements from entire elements disclosed in the respective embodiments and the modified examples may be possible, and also formation by suitably combining the elements of a different embodiment and a modified example may be possible.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus provided in an imaging system having an image capturing unit, the image capturing unit being configured to irradiate a subject with light and to generate first image data representing a first image based on the light reflected from the subject and having first spectral characteristics, and to generate second image data representing a second image based on the light reflected from the subject and having second spectral characteristics different from the first spectral characteristics, the image processing apparatus being configured to perform image processing on the first image and the second image, the image processing apparatus comprising:
a computing unit configured to determine a degree of correlation between the first image and the second image; and
a control unit configured to cause the image capturing unit to generate the first image data at a preset frame rate, and configured to control timing to generate the second image data instead of the first image data based on a determination result of the degree of correlation.

2. The image processing apparatus according to claim 1, wherein
the light having the second spectral characteristics has a limited wavelength band relative to the light having the first spectral characteristics.

3. The image processing apparatus according to claim 1, wherein
the computing unit comprises:
a correlation calculation unit configured to calculate a parameter indicating the degree of correlation between the first image and the second image; and
a correlation determination unit configured to determine whether there is correlation between the first image and the second image, by comparing the parameter with a threshold, and
the control unit is configured to cause the image capturing unit to generate the second image data if it is determined that there is no correlation between the first image and the second image.

4. The image processing apparatus according to claim 1, wherein
the computing unit comprises:
a region extraction unit configured to extract a region of interest from the second image; and
a tracking determination unit configured to determine whether the region of interest extracted from the second image can be tracked in the first image, and
the control unit is further configured to cause the image capturing unit to generate the second image data if the region of interest cannot be tracked in the first image.

5. The image processing apparatus according to claim 4, wherein
the computing unit further comprises:
a region storage unit configured to store the region of interest; and
a region deformation processing unit configured to deform the region of interest so as to conform to a corresponding region in the first image if it is determined that the region of interest can be tracked in the first image,
the region storage unit is configured to sequentially update and store the region of interest deformed by the region deformation processing unit, and
the tracking determination unit is configured to determine whether the region of interest stored in the region storage unit can be tracked in the first image.

6. The image processing apparatus according to claim 1, wherein
the control unit is further configured to cause the image capturing unit to generate the second image data when the first image data is continuously generated a predetermined number of times or more.

7. The image processing apparatus according to claim 1, further comprising an input unit configured to input a command signal to the control unit in accordance with operation from outside, wherein
the control unit is further configured to cause the image capturing unit to generate the second image data when the command signal is received from the input unit.

8. The image processing apparatus according to claim 1, wherein
the control unit is configured to cause the image capturing unit to perform a set of operations to generate the second image data multiple times based on a plurality of kinds of light having spectral characteristics different from the first spectral characteristics and also different from one another.

9. The image processing apparatus according to claim 8, wherein
in the set of operations, an operation of generating the first image data is inserted at least once between operations of generating the second image data multiple times.

10. The image processing apparatus according to claim 1, further comprising a display unit configured to display the first image and the second image side by side.

11. The image processing apparatus according to claim 1, further comprising a display unit configured to:
display the first image in a first area on a screen; and
display, in an area other than the first area on the screen, at least one thumbnail image obtained by reducing the second image.

12. The image processing apparatus according to claim 5, further comprising a display unit configured to:
display the first image; and
highlight a region in the first image corresponding to the region of interest stored in the region storage unit.

13. The image processing apparatus according to claim 5, further comprising a display unit configured to:
display the first image; and
superimpose an image of the region of interest stored in the region storage unit on the first image to display the superimposed image.

14. An endoscope system comprising:
the image processing apparatus according to claim 1; and
the image capturing unit.

15. The endoscope system according to claim 14, wherein
the image capturing unit comprises:
a light source configured to generate white light;
an image sensor configured to receive light reflected from the subject and to generate an imaging signal; and
a wavelength selecting unit disposed between the light source and the subject.

16. The endoscope system according to claim 14, wherein
the image capturing unit comprises:
a first light source configured to generate light having the first spectral characteristics;
a second light source configured to generate light having the second spectral characteristics; and
an image sensor configured to receive light reflected from the subject and to generate an imaging signal.

17. The endoscope system according to claim 14, wherein
the image capturing unit comprises:
a light source configured to generate white light;
an image sensor configured to receive light reflected from the subject and to generate an imaging signal; and
a wavelength selecting unit disposed between the subject and the image sensor.

18. An image processing method, comprising:
irradiating a subject with light and generating image data representing a first image based on the light reflected from the subject and having first spectral characteristics;
irradiating the subject with light and generating image data representing a second image based on the light reflected from the subject and having second spectral characteristics different from the first spectral characteristics;
determining a degree of correlation between the first image and the second image; and
causing the first image data to be generated at a preset frame rate, and controlling timing to generate the second image data instead of the first image data based on a determination result of the degree of correlation.

19. A non-transitory computer-readable recording medium with an executable image processing program stored thereon, the program causing a computer to execute:
irradiating a subject with light and generating image data representing a first image based on the light reflected from the subject and having first spectral characteristics;
irradiating the subject with light and generating image data representing a second image based on the light reflected from the subject and having second spectral characteristics different from the first spectral characteristics;
determining a degree of correlation between the first image and the second image; and
causing the first image data to be generated at a preset frame rate, and controlling timing to generate the second image data instead of the first image data based on a determination result of the degree of correlation.
